(19) United States
(12) Patent Application Publication
(10) Pub. No.: US 2026/0199030 A1
(43) Pub. Date: Jul. 16, 2026

(54) AUGMENTED REALITY SIMULATED SETUP AND CONTROL OF ROBOTIC SURGICAL SYSTEMS WITH INSTRUMENT OVERLAYS

(71) Applicant: Covidien LP, Mansfield, MA (US)

(72) Inventors: Michael A. Eiden, Somerville, MA (US); Max L. Balter, Boston, MA (US); Tuvia C. Rappaport, Somerville, MA (US); Zachary A. Walker-Liang, Kensington, MD (US)

(21) Appl. No.: 19/136,009

(22) PCT Filed: Dec. 13, 2023
(86) PCT No.: PCT/IB2023/062624

§ 371 Date: Jun. 5, 2025

Related U.S. Application Data
(60) Provisional application No. 63/432,431, filed on Dec. 14, 2022.

(51) Int. Cl. A61B 34/00; A61B 34/20; A61B 34/37; A61B 90/00; G06V 10/70; G06V 20/20

(52) U.S. Cl. CPC A61B 34/25; A61B 34/37; G06V 10/70; G06V 20/20; A61B 2034/2065; A61B 2090/0805; A61B 2090/365; G06V 2201/034

(57) ABSTRACT

A system for workspace augmentation includes an augmented reality device. The augmented reality headset includes an imaging device configured to capture images of a real-world environment, a display configured to display a composite view, a processor, and a memory. The memory includes instructions stored thereon, which, when executed by the processor, cause the system to: capture a real-world environment that includes an object by the imaging device, identify the object in the captured image, determine information relating to the object, render an overlay including the information relating to the object, and display the information relating to the object on the display. The object includes a surgical instrument of a robotic surgical system.

This application claims the benefit of U.S. Provisional Patent Application Ser. No. 63/432,431, filed Dec. 14, 2022, the entire content of which is incorporated herein by reference.

### BACKGROUND

### Technical Field

The disclosure generally relates to systems and methods for workspace augmentations. In particular, the present disclosure is directed to an augmented reality simulated setup of robotic surgical systems with instrument overlays.

### Background of Related Art

Robotic surgical systems are currently being used in minimally invasive medical procedures. Some robotic surgical systems include a surgical console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector at a worksite within the patient's body.

### SUMMARY

In accordance with aspects of the disclosure, a computer-implemented method for workspace simulation is presented. The method includes capturing a real-world environment by an imaging device. The real-world environment includes an object. The object includes a surgical instrument of a robotic surgical system. The method further includes identifying the object in the captured real-world environment, determining information relating to the object, rendering an overlay including the information relating to the object, and displaying the information relating to the object on a display of an augmented reality device, wherein the display is configured to display a composite view.

In an aspect of the disclosure, the displayed information may include a use life of the object, a quantity of uses remaining, a number of times used, a time the object was used for, total forces, maximum forces, name, serial number, batch number, lot number, expiration date, and/or total time of delivering energy versus total time the object is used for.

In another aspect of the disclosure, identifying the object in the captured real-world environment may be based on object detection.

In yet another aspect of the disclosure, object detection may be performed by generating a spatial mesh based on the captured real-world environment; determining boundaries of the object based on the spatial mesh; and identifying the object based on a machine learning model, where the determined boundaries are provided as an input to the machine learning model.

In a further aspect of the disclosure, identifying the object in the captured real-world environment may be based on identifying a machine-readable identifier of the object, comparing the machine-readable identifier to a predetermined database of machine-readable identifiers associated with objects, and identifying the object based on the comparison.

In yet a further aspect of the disclosure, identifying the object in the captured real-world environment may be based on receiving a wireless signal from the object, where the wireless signal includes information; and identifying the object based on the information included in the wireless signal.

In yet a further aspect of the disclosure, the method may further include receiving a command to display an object dashboard and displaying on the display the object dashboard.

In another aspect of the disclosure, the object dashboard may include an object history, a current object state, and/or object use instructions.

In yet a further aspect of the disclosure, the method may further include determining that the object is inserted in an abdomen of a patient, wherein the identified object includes a surgical port, and displaying on the display information relating to the surgical port based on the determination.

In another aspect of the disclosure, the method may further include displaying a prompt indicating instructions for replacing a reload and/or a stapling cartridge of a surgical instrument.

In accordance with aspects of the disclosure, a system for workspace augmentation that includes an augmented reality device (e.g., an AR headset) is presented. The augmented reality headset includes an imaging device configured to capture images of a real-world environment, a display configured to display a composite view, a processor, and a memory. The memory includes instructions stored thereon, which, when executed by the processor, cause the system to: capture an image of a real-world environment that includes an object by the imaging device, identify the object in the captured image, determine information relating to the object, render an overlay including the information relating to the object, and display the information relating to the object on the display. The object includes a surgical instrument of a robotic surgical system.

In an aspect of the disclosure, the displayed information may include a use life of the object, a quantity of uses remaining, a number of times used, a time the object was used for, total forces, maximum forces, name, serial number, batch number, lot number, expiration date, and/or total time of delivering energy versus total time the object is used for.

In another aspect of the disclosure, the identifying the object in the captured image may be based on object detection.

In yet another aspect of the disclosure, the object detection may be performed by generating a spatial mesh based on the captured real-world environment, determining boundaries of the object based on the spatial mesh, and identifying the object based on a machine learning model, where the determined boundaries are provided as an input to the machine learning model.

In a further aspect of the disclosure, the identifying the object in the captured image may be based on identifying a machine-readable identifier of the object, comparing the machine-readable identifier to a predetermined database of machine-readable identifiers associated with objects, and identifying the object based on the comparison.

In a further aspect of the disclosure, the identifying the object in the captured image may be based on receiving a wireless signal from the object, where the wireless signal includes information; and identifying the object based on the information included in the wireless signal.

In yet a further aspect of the disclosure, the instructions, when executed by the processor, may further cause the system to receive a command to display an object dashboard and display on the display the object dashboard.

In an aspect of the disclosure, the object dashboard may include an object history, a current object state, and/or object use instructions.

In another aspect of the disclosure, the instructions, when executed by the processor, may further cause the system to display a prompt indicating instructions for replacing at least one of a reload and/or a stapling cartridge of a surgical instrument.

In accordance with aspects of the disclosure, a non-transitory computer-readable medium is presented. The non-transitory computer-readable medium stores instructions which, when executed by a processor, cause the processor to perform a method that includes capturing a real-world environment by an imaging device, where the real-world environment includes an object, identifying the object in the captured real-world environment, determining information relating to the object, rendering an overlay including the information relating to the object, and displaying the information relating to the object on a display of an augmented reality headset. The object includes a surgical instrument of a robotic surgical system

### BRIEF DESCRIPTION OF THE DRAWINGS

Various aspects of the disclosure are described herein with reference to the drawings wherein:

FIG. 1 is a schematic illustration of a robotic surgical system including a control tower, a console, and one or more surgical robotic arms according to an aspect of the disclosure;

FIG. 2 is a perspective view of a surgical robotic arm of the robotic surgical system of FIG. 1 according to an aspect of the disclosure;

FIG. 3 is a perspective view of a setup arm with the surgical robotic arm of the robotic surgical system of FIG. 1 according to an aspect of the disclosure;

FIG. 4 is a schematic diagram of a computer architecture of the robotic surgical system of FIG. 1 according to an aspect of the disclosure;

FIG. 5 is a schematic view of the robotic surgical system of FIG. 1 positioned about a surgical table according to an embodiment of the present disclosure;

FIG. 6 is a flow chart for a computer-implemented method for workspace augmentation according to an aspect of the disclosure;

FIG. 7 is an image of a composite view of the workspace augmentation with a surgical instrument with an overlay according to an aspect of the disclosure; and

FIG. 8 is an image of a composite view of the workspace augmentation with a surgical port with an overlay according to an aspect of the disclosure.

### DETAILED DESCRIPTION

Aspects of the presently disclosed robotic surgical system are described in detail with reference to the drawings, in which like reference numerals designate identical or corresponding elements in each of the several views. As used herein, the term “distal” refers to the portion of the robotic surgical system and/or the surgical instrument coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller or on a user device, including, for example, a mobile device, a personal computer, or a server system.

As will be described in detail below, the disclosure is directed to a robotic surgical system, which includes a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which would, in turn, move the robotic arm in response to the movement command.

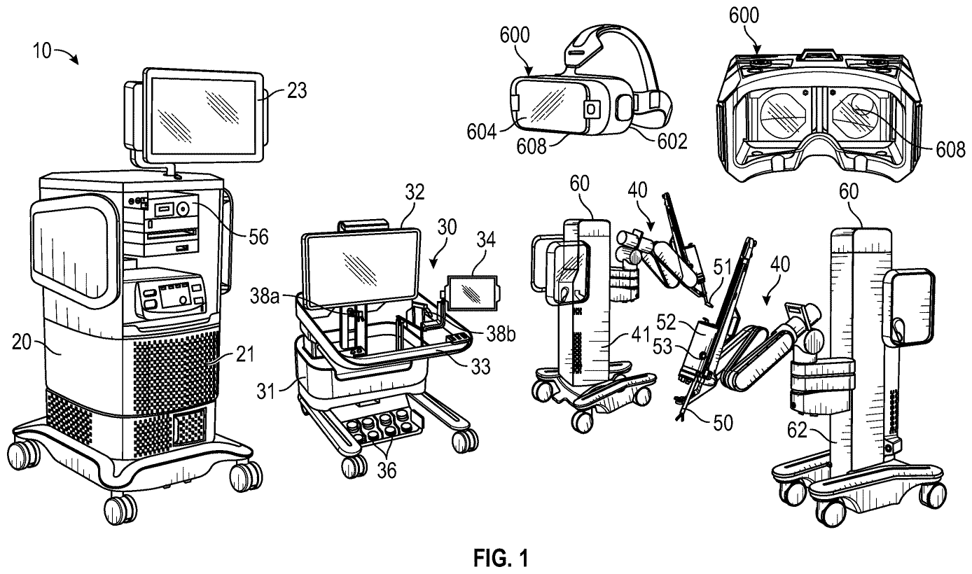

With reference to FIG. 1, a robotic surgical system 10 generally includes an augmented reality headset 600, a control tower 20, which is connected to all of the components of the robotic surgical system 10, including a surgical console 30 and one or more robotic arms 40. Each of the robotic arms 40 includes a surgical instrument 50 removably coupled thereto. Each of the robotic arms 40 is also coupled to a movable cart 60.

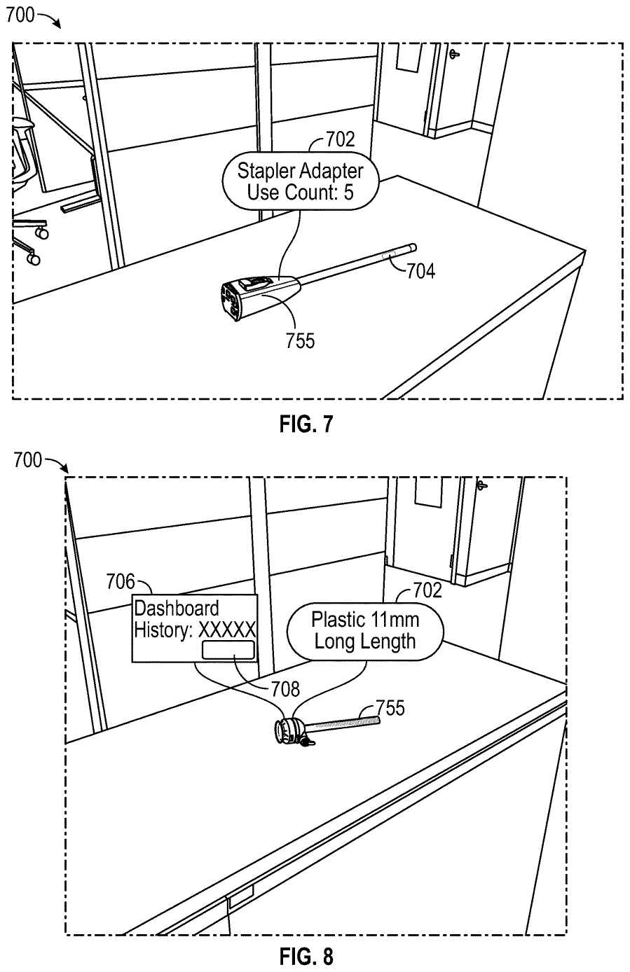

The augmented reality headset 600 configured to display a composite view generally includes a controller 602, an imaging device 604, and a display 608. The controller 602 includes a memory configured to have instructions stored thereon and a processor configured to execute the instructions. The augmented reality headset 600 may overlay virtual objects such as a virtual robot arm (FIG. 7). For example, the augmented reality headset 600 can provide users advice on how to position various virtual objects to help set up an operating room for a surgery. It is contemplated that the augmented reality headset 600 may be full virtual reality such as the Quest 2® from Meta®, of Menlo Park, CA or an augmented reality (mixed reality) headset such as Hololens® from Microsoft®, of Seattle, WA.

The surgical instrument 50 is configured for use during minimally invasive surgical procedures. In aspects, the surgical instrument 50 may be configured for open surgical procedures. In aspects, the surgical instrument 50 may be an endoscope, such as an endoscopic camera 51, configured to provide a video feed for the user. In further aspects, the surgical instrument 50 may be an electrosurgical forceps configured to seal tissue by compressing tissue between jaw members and applying electrosurgical current thereto. In yet further aspects, the surgical instrument 50 may be a surgical stapler including a pair of jaws configured to grasp and clamp tissue while deploying a plurality of tissue fasteners, e.g., staples, and cutting stapled tissue.

One of the robotic arms 40 may include the endoscopic camera 51 configured to capture video of the surgical site. The endoscopic camera 51 may be a stereoscopic endoscope configured to capture two side-by-side (i.e., left and right) images of the surgical site to produce a video stream of the surgical scene. The endoscopic camera 51 is coupled to a video processing device 56, which may be disposed within the control tower 20. The video processing device 56 may be any computing device as described below configured to receive the video feed from the endoscopic camera 51 perform the image processing based on the depth estimating algorithms of the disclosure and output the processed video stream. Processing done on the video feed from the endoscopic camera 51 may be turned into valuable information to display on an overlay showing an augmented reality instrument label. For example, the video feed from the endoscopic camera 51 may be processed and an augmented reality instrument label may be displayed indicating whether or not the surgical instrument 50 is safe to withdraw, or for example, if the surgical instrument 50 is still clutching tissue.

The surgical console 30 includes a first display 32, which displays a video feed of the surgical site provided by camera 51 of the surgical instrument 50 disposed on the robotic arms 40, and a second display 34, which displays a user interface for controlling the robotic surgical system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support the user's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

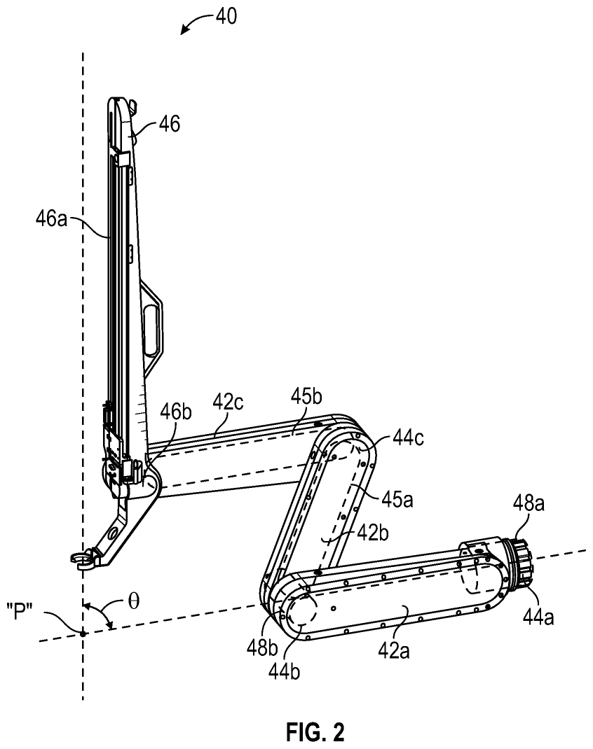

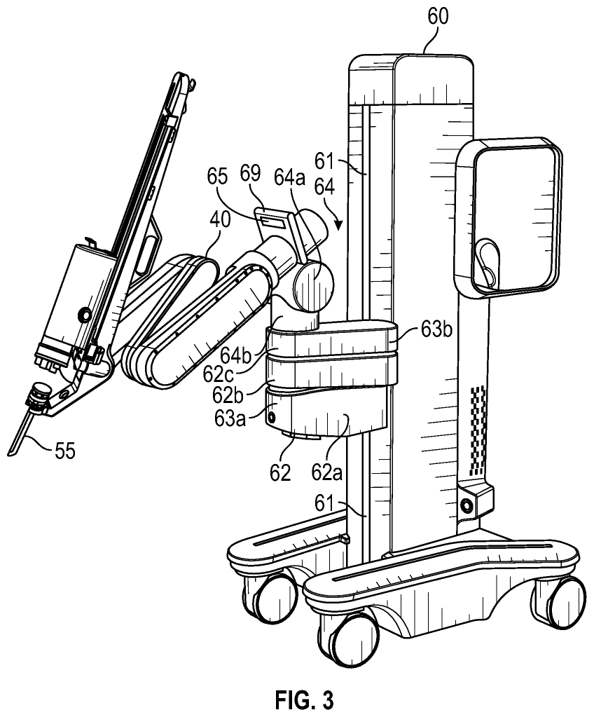

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48 b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In aspects, some, or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the robotic arm 40 also includes a holder 46 defining a second longitudinal axis and configured to receive an instrument drive unit (IDU) 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effector) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During endoscopic procedures, the instrument 50 may be inserted through an access port 55 (FIG. 3) held by the holder 46.

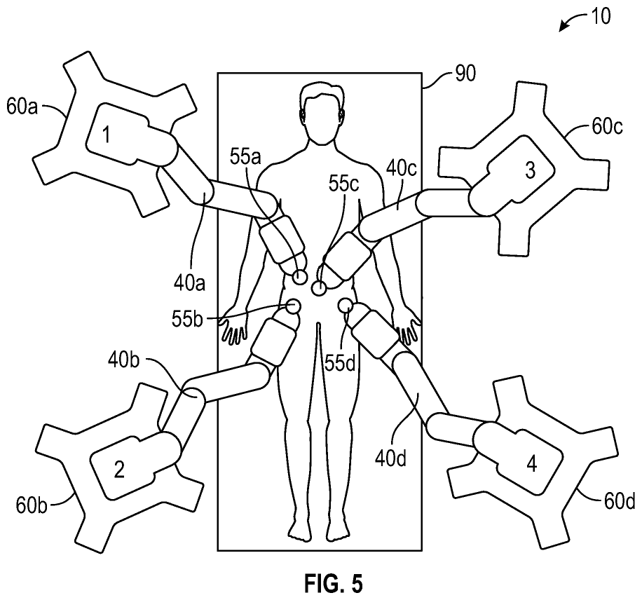

The robotic arm 40 also includes a plurality of manual override buttons 53 (FIGS. 1 and 5) disposed on the IDU 52 and the setup arm 62, which may be used in a manual mode. The user may press one or more of the buttons 53 to move the component associated with the button 53.

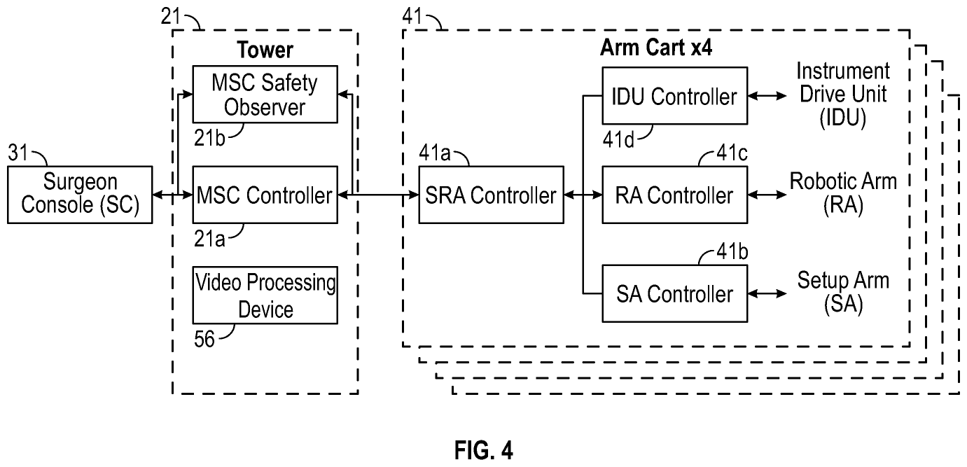

With reference to FIG. 4, each of the computers 21, 31, 41 of the robotic surgical system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the robotic surgical system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the handle controller controlling the robotic arm 40, e.g., the handle controller 38a, which is transformed into a desired pose of the robotic arm 40 through a hand-eye transform function executed by the controller 21a. The hand-eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

The video processing device 56 is configured to process the video feed from the endoscope camera 51 and to output a processed video stream on the first displays 32 of the surgical console 30 and/or the display 23 of the control tower 20.

With reference to FIG. 5, the robotic surgical system 10 is setup around a surgical table 90. The system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert instruments 50 and the laparoscopic camera 51 into corresponding ports 55a-d.

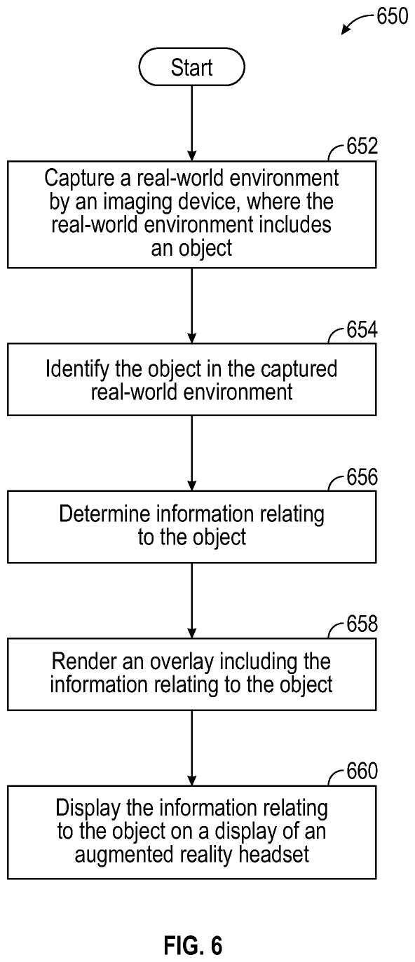

FIG. 6 shows a flow chart illustrating the various operations of an exemplary method for workspace augmentation with augmented reality instrument overlays. Persons skilled in the art will appreciate that one or more operations of the method 650 may be performed in a different order, repeated, and/or omitted without departing from the scope of the disclosure. In various aspects, the illustrated method 650 can operate in controller 602 (FIG. 1), in a remote device, or in another server or system. Other variations are contemplated to be within the scope of the disclosure. The operations of method 650 will be described with respect to a controller, e.g., controller 602 (FIG. 1) of augmented reality headset 600 (FIG. 1), but it will be understood that the illustrated operations are applicable to other systems and components thereof as well.

Initially, at step 652, the controller 602 causes the robotic surgical system 10 to capture a real-world environment by an imaging device 604 of an augmented reality headset 600 (FIG. 1) (or a mobile device/tablet). The imaging device 604 may include a stereoscopic imaging device. The real-world environment includes an object 55. The object 55 may be the surgical instrument 50, the access port 55, or any other instrument or accessory of the robotic surgical system 10. In aspects, the controller 602 may render a 3-D representation of the captured real-world.

Next, at step 654, the controller 602 causes the robotic surgical system 10 to identify the object 755 in the captured real-world environment. For example, the robotic surgical system 10 may identify the object 755 as a surgical instrument adapter or a surgical port (FIG. 7). The surgical object 755 may be identified using image-based object identification, a machine-readable identifier marker, machine-readable identifier (e.g., barcode or machine-readable identifier marker), wireless identifier (e.g., RFID). In aspects, the controller 602 may enable manual registration and/or detection of a machine-readable identifier/marker on an object 755.

In aspects, identifying the object 755 in the captured real-world environment may be based on object detection. The object detection may be performed by generating a spatial mesh based on the captured real-world environment, determining boundaries of the object based on the spatial mesh, and identifying the object 755 based on a machine learning model (e.g., a convolutional neural network). The determined boundaries may be provided as an input to the machine learning model. The machine learning model may be trained on labeled images of objects, e.g., surgical instruments.

In aspects, identifying the object in the captured real-world environment may be based on identifying a machine-readable identifier of the object, comparing the machine-readable identifier 704 to a predetermined database of machine-readable identifiers associated with objects, and identifying the object based on the comparison.

In aspects, identifying the object in the captured real-world environment may be based on receiving a wireless signal from the object, where the wireless signal includes information, and identifying the object based on the information included in the wireless signal.

Next, at step 656, the controller 602 causes the robotic surgical system 10 to determine information relating to the object 755. The information may include a use life of the object, a quantity of uses remaining, a number of times used, a time the object was used for, total forces, maximum forces, name, serial number, batch number, lot number, expiration date, total time of delivering energy versus total time the object is used for, and/or other relevant information.

At step 658, the controller 602 causes the robotic surgical system 10 to rendering an overlay 702 including the information relating to the object (FIG. 7), which includes information overlaid on the real-world environment. The composite view 700 may represent a clinical workspace simulation, which may be used, for example, to guide staff in setting up a robotic surgical system 10 based on the information relating to the object 755. In another example, the composite view 700 may represent a clinical workspace augmentation, which may be used to guide tasks intraoperatively. FIG. 7 is an image of a composite view 700 of the clinical workspace augmentation with a surgical instrument with an overlay 702. For example, the overlay 702 may display information such as what the identified object 755 is (“Stapler Adapter”) and/or other information relating to the object 755, such as the number of uses (e.g., “Use Count: 5”).

Next, at step 660, the controller 602 causes the robotic surgical system 10 to display the information relating to the object 755 on the display 608 of the augmented reality headset 600 (FIG. 1) configured to display the composite view. For example, the displayed information may include, a quantity of uses remaining, and a number of times used. This provides the benefit of not having to plug in (i.e., connect) the surgical instrument to the robotic surgical system 10, which may be detected as a “usage” count, thereby inadvertently decreasing usable life of the instrument 50 or other accessory. In aspects, the displayed overlay may move with the object 755 if the object 755 is moved.

The controller 602 may also suggest optimal placement of the object 755 based on its function and the type of surgery. In aspects, the controller 602 may display the composite view 700 on a user device, such as a mobile device and/or a tablet, the display 608 of the augmented reality headset 600, or on one of the displays 23, 32, 34 of the robotic system 10.

In aspects, the controller 602 may receive a command to display an object dashboard 706, and display on the display the object dashboard 706 (FIG. 8). The object dashboard 706 may include an object history, object state (e.g., “Expired” “Grasping Tissue” and/or “Straight”), and/or object use instructions and other information providing relevant information to the staff. In aspects, the controller 602 may be synced with an inventory management system, and enable the display of quantity available and/or an option to request inventory, for example additional reloads. FIG. 8 is an image of a composite view 700 of the clinical workspace augmentation with a surgical port with the overlay 702. The composite view 700 may include a dashboard 706 that shows information relating to the object 755, such as a use history of the object 755. The overlay 702 may also indicate information relating to the type of object 755 such as (e.g., “Plastic 11 mm” and “Long Length”). The dashboard may include a button 708 for toggling the display of the dashboard 706 on or off.

In aspects, the controller 602 may cause the robotic surgical system 10 to display a prompt indicating instructions for replacing at least one of a reload and/or a stapling cartridge of the surgical instrument. For example, the controller 602 may cause the robotic surgical system 10 to capture the image of the surgical instrument. The controller 602 may cause the robotic surgical system 10 to render the overlay 702 that has the information relating to the object (FIG. 7) overlaid on the real-world environment. The overlay may display, for example, the prompt indicating instructions for replacing a reload of the surgical instrument. The prompt may be displayed on the display 608 of the augmented reality headset 600 (FIG. 1). I aspects, the overlay may provide guidance the user on how to connect cables from a generator to the surgical instrument.

In aspects, the controller 602 may detect a patient (or a clinician) in the real-world environment by the imaging device and display the detected patient by a display 608 (FIG. 1) of the augmented reality headset 600. Real-world objects, such as the patient, the user, and/or the surgical table 90, may be detected using edge detection and/or image segmentation. For example, the controller 602 may extract the edges of the object 755 in the captured image, by detecting discontinuities in depth, discontinuities in surface orientation, and/or changes in material properties of the object 755 in the captured image. The extracted edges may be used to determine the boundaries of the object 755. The controller may then identify the object 755 based on the determined boundaries, for example, as the surgical instrument or as the clinician.

In aspects, the controller 602 may cause the robotic surgical system 10 to determine that the object is inserted in an abdomen of a patient. For example, the identified object 755 may be the access port (FIG. 8). The controller 602 may cause the robotic surgical system 10 to display on the display information relating to the surgical port based on the determination. For example, the overlay 702 may indicate a type of surgical port when inserted in a patient's abdomen (e.g., metal vs. plastic, diameter, length, etc.). In aspects, the controller 602 may explicitly differentiate between plastic vs. metal ports, length, and diameter. For example, the controller 602 may detect which arm the port is connected to, e.g., RH, LH, ENDO, RES.

In aspects, the controller 602 may cause the robotic surgical system 10 provide a visualization of a sterile field for the user by the surgical table 90. This provides the benefit of enabling the user (e.g., a clinician) to see what is sterile and what is not sterile. The visualization of a sterile field may include a color, gradient, and/or shading. For example, the controller 602 may cause the robotic surgical system 10 to display on the display 608 a red shaded area indicating where the surgical table 90 is not sterile. In another example, the sterile field may be shown as green.

During port placement in a procedure, the controller 602 may cause the robotic surgical system 10 to generate an overlay 702 that displays measurements overlaid on the surgical port, umbilicus, and/or other structure, for example, in response to the curvature of an insufflated abdomen of a patient. For example, the controller 602 may access measurements based on the insufflated abdomen of a patient and in response to the measurements, display the measurements overlaid on the surgical port.

The controller 602 may cause the robotic surgical system 10 to generate an overlay displaying virtual monitors to display an endoscope video in real-time on the display 608 of the augmented reality headset 600 (FIG. 1). In aspects, the disclosed technology may be extended to control robotic arm motion. For example, wearing an augmented reality headset 600 (FIG. 1), the user would be able to visualize the endoscope feed directly in-front of them with the ability to toggle between a 2D/3D visualization, adjust the scale, position, and/or rotation of the visualization. In aspects, the augmented reality headset 600 may enable the user to tune display settings, such as brightness and/or contrast of the visualization.

The controller 602 may provide enhanced feedback to the clinical staff by overlaying information on the composite view 700, such as recommended surgical port entry points on a patient's abdomen. The surgical port entry point may be based on a body habitus of the patient. In aspects, the controller 602 may render real-time measurements or suggestions of the surgical port entry points based on the patient body habitus for different locations on the patient. For example, the controller 602 may display an indication that the surgical port should be about 5 cm above and about 5 cm to the left of the naval.

It will be understood that various modifications may be made to the aspects disclosed herein. Therefore, the above description should not be construed as limiting but merely as exemplifications of various aspects. Those skilled in the art will envision other modifications within the scope and spirit of the claims appended thereto.

What is claimed is:

1. A computer-implemented method for workspace augmentation, the method comprising:
capturing a real-world environment by an imaging device, wherein the real-world environment includes an object, wherein the object includes a surgical instrument of a robotic surgical system;
identifying the object in the captured real-world environment;
determining information relating to the object;
rendering an overlay including the information relating to the object; and
displaying the information relating to the object on a display of an augmented reality device, wherein the display is configured to display a composite view.

2. The computer-implemented method of claim 1, wherein the displayed information includes at least one of a use life of the object, a quantity of uses remaining, a number of times used, a time the object was used for, total forces, maximum forces, name, serial number, batch number, lot number, expiration date, or total time of delivering energy versus total time the object is used for.

3. The computer-implemented method of claim 1, wherein the identifying the object in the captured real-world environment is based on object detection.

4. The computer-implemented method of claim 3, wherein the object detection is performed by:
generating a spatial mesh based on the captured real-world environment;
determining boundaries of the object based on the spatial mesh; and
identifying the object based on a machine learning model, where the determined boundaries are provided as an input to the machine learning model.

5. The computer-implemented method of claim 1, wherein the identifying the object in the captured real-world environment is based on:
identifying a machine-readable identifier of the object;
comparing the machine-readable identifier to a predetermined database of machine-readable identifiers associated with objects; and
identifying the object based on the comparison.

6. The computer-implemented method of claim 1, wherein the identifying the object in the captured real-world environment is based on:
receiving a wireless signal from the object, wherein the wireless signal includes information; and
identifying the object based on the information included in the wireless signal.

7. The computer-implemented method of claim 1, further comprising:
receiving a command to display an object dashboard; and
displaying on the display the object dashboard.

8. The computer-implemented method of claim 7, wherein the object dashboard includes an object history, a current object state, or object use instructions.

9. The computer-implemented method of claim 1, further comprising:
determining that the object is inserted in an abdomen of a patient, wherein the identified object includes a surgical port, and displaying on the display information relating to the surgical port based on the determination.

10. The computer-implemented method of claim 1, further comprising:
displaying a prompt indicating instructions for replacing at least one of a reload or a stapling cartridge of the surgical instrument.

11. A system for workspace augmentation, the system comprising:
an augmented reality headset including:
an imaging device configured to capture images of a real-world environment;
a display configured to display a composite view;
a processor; and
a memory, including instructions stored thereon, which, when executed by the processor, cause the system to:
capture an image of the real-world environment by the imaging device, wherein the real-world environment includes an object, wherein the object in the captured image includes a surgical instrument of a robotic surgical system;
identify the object in the captured image;
determine information relating to the object;
render an overlay including the information relating to the object; and
display the information relating to the object on the display.

12. The system of claim 11, wherein the displayed information includes at least one of a use life of the object, a quantity of uses remaining, a number of times used, a time the object was used for, total forces, maximum forces, name, serial number, batch number, lot number, expiration date, and/or total time of delivering energy versus total time the object is used for.

13. The system of claim 11, wherein the identifying the object in the captured image is based on object detection.

14. The system of claim 13, wherein the object detection is performed by:
generating a spatial mesh based on the captured real-world environment;
determining boundaries of the object based on the spatial mesh; and
identifying the object based on a machine learning model, where the determined boundaries are provided as an input to the machine learning model.

15. The system of claim 11, wherein the identifying the object in the captured image is based on:
identifying a machine-readable identifier of the object;
comparing the machine-readable identifier to a predetermined database of machine-readable identifiers associated with objects; and
identifying the object based on the comparison.

16. The system of claim 11, wherein the identifying the object in the captured image is based on:
receiving a wireless signal from the object, wherein the wireless signal includes information; and
identifying the object based on the information included in the wireless signal.

17. The system of claim 11, wherein the instructions, when executed by the processor, further cause the system to:
receive a command to display an object dashboard; and
display on the display the object dashboard.

18. The system of claim 17, wherein the object dashboard includes an object history, a current object state, or object use instructions.

19. The system of claim 11, wherein the instructions, when executed by the processor, further cause the system to:
display a prompt indicating instructions for replacing at least one of a reload or a stapling cartridge of a surgical instrument.

20. A non-transitory computer-readable medium storing instructions which, when executed by a processor, cause the processor to perform a method comprising:
capturing a real-world environment by an imaging device, wherein the real-world environment includes an object;
identifying the object in the captured real-world environment;
determining information relating to the object;
rendering an overlay including the information relating to the object; and
displaying the information relating to the object on a display of an augmented reality headset.
